(19) United States
(12) Patent Application Publication
(10) Pub. No.: US 2007/0059736 A1
(43) Pub. Date: Mar. 15, 2007

(54) Bio-analysis chip, bio-analysis system and bio-analysis method

(75) Inventors: Hisao Saito, Matsudo (JP); Yasuhiko Sasaki, Tsuchiura (JP); Ryo Miyake, Tsukuba (JP)

Correspondence Address: MATTINGLY, STANGER, MALUR & BRUNDIDGE, P.C., 1800 DIAGONAL ROAD, SUITE 370, ALEXANDRIA, VA, 22314, US

(73) Assignee: Hitachi, Ltd., Tokyo (JP)

(21) Appl. No.: 11/450,366

(22) Filed: Jun. 12, 2006

(30) Foreign Application Priority Data
Jun. 14, 2005 (JP) 2005-173326

(51) Int. Cl. C12Q 1/68; C12M 1/34

(52) U.S. Cl. 435/6; 435/287.2

(57) ABSTRACT

A bio-analysis chip which is convenient in use and can promptly detect microbes with high accuracy. The bio-analysis chip comprises a collection member for collecting microbes in the atmosphere with the microbes adhering to the collection member, and a substrate in the form of a thin plate on which the collection member is mounted. The substrate has a collection member receiving section in which the collection member is placed, and a plurality of reagent reservoirs in which reagents for treating and analyzing the microbes are stored. The plurality of reagent reservoirs are connected to the collection member receiving section.

### BACKGROUND OF THE INVENTION

1. Field of the Invention

The present invention relates to a bio-analysis chip, a bio-analysis system, and a bio-analysis method. More particularly, the present invention relates to a bio-analysis chip, a bio-analysis system and a bio-analysis method which are suitable for collecting microbes floating in the atmosphere and for analyzing the collected microbes.

2. Description of the Related Art

JP,A 2000-125843 (Patent Document 1) discloses one example of known portable air-floating germ samplers for collecting air-floating germs in a room. The disclosed portable air-floating germ sampler is used to examine and manage the contamination status attributable to microbes, etc. floating in the atmosphere. The portable air-floating germ sampler comprises a nozzle having many holes, a nozzle holder for holding the nozzle, a petri dish (Schale) support for supporting a petri dish which is positioned downstream of the nozzle and contains a culture medium, and a fan for forming an air flow. With the portable air-floating germ sampler, microbes can be collected on the culture medium by sucking the atmosphere such that the microbes collide against the culture medium supported on the petri dish at high speed.

Also, JP,A 2005-65607 (Patent Document 2) discloses a known small-sized portable analysis apparatus including an analysis chip which is easy to handle, is inexpensive, and is capable of automating a series of steps from supply of a sample to extraction and analysis of a gene in a continuous manner. A gene treatment chip used in the analysis apparatus comprises an inlet port to which is supplied the sample containing the gene, a solvent storage section for storing a solvent introduced to the sample that has been supplied to the inlet port, a gene extracting section to which is introduced a mixed solution of the sample and the solvent and which includes a gene binding carrier which is bound to the gene, a washing liquid storage section for storing a washing liquid introduced to the gene extracting section, an eluent storage section for storing an eluent introduced to the gene extracting section, and a reaction section to which is introduced the gene eluted by the eluent.

An analysis method performed by the disclosed analysis apparatus is as follows. First, a solution containing a chaotropic ion is mixed in a sample for lysis of a virus or cell membrane surrounding a target gene by the action of the chaotropic ion. Then, silica is added to the mixture after the lysis so that the gene and the silica are specifically bound to each other by the action of the chaotropic ion. The gene-silica bound substance is washed by ethanol having a high concentration to remove protein and the chaotropic ion which are contained in the sample. After the washing, water or a solution having a low salt concentration is added to the gene-silica bound substance, thus causing the gene to elute from the silica. A primer, DNA synthesis enzymes, 4 kinds of substrates (dNTP), etc. are added to the eluted gene and a temperature cycle of “heat denaturation—annealing—synthesis of complementary strands” is applied to the gene, thereby amplifying the gene. The amplification of the gene can be detected in real time by injecting a fluorescent dye together with the above-mentioned reagents in advance and by applying the temperature cycle while excitation light is irradiated.

### SUMMARY OF THE INVENTION

The above-cited Patent Document 1 discloses the portable air-floating germ sampler for collecting microbes on the culture medium supported on the petri dish, but it does not disclose a method for detecting the microbes collected on the culture medium. In consideration of that the microbes are collected on the culture medium supported on the petri dish, Patent Document 1 seems to employ, as an analysis method, the so-called cultivation method of cultivating the microbes on the culture medium and observing whether the microbes proliferate. The case of employing such an analysis method accompanies with the problem that a long period of 2-7 days is usually required to cultivate the microbes.

Recently, another gene analysis method has also been practiced which comprises the steps of amplifying a gene of a target microbe, detecting the amplified gene, and determining the presence or absence of the target microbe in a shorter period. For example, when germs, such as Bacillus anthracis and Bacillus cereus, are brought into a situation where water is hardly contained in the surroundings and nutrient is exhausted, the germs form pores in several hours. Because the pores are very hard shell-like substances and have strong resistance against heat, chemical materials, ultraviolet rays, etc., proper treatment for removing the pores is required. Up to now, a step of the pore treatment and a step of extracting the gene from the germ have been all performed by manual operations. Therefore, the operations are very intricate and an inspection operator is limited to a skilled person while accompanying a risk of infection by the inspection operator. Further, reaction cuvettes, pipettes, etc. used in the manual operations become all infected wastes, thus leading to a risk of secondary contamination. Another drawback is that the manual operations cause instability in analysis accuracy.

On the other hand, in the gene treatment chip used in the analysis apparatus disclosed in Patent Document 2, the sample containing the gene is supplied to the inlet port. Accordingly, the gene treatment chip is not used from the stage of collecting the sample, and hence it does not pay consideration to convenience in use for a process including the transition from the sample collecting step to the gene treating step.

Another problem with Patent Document 2 is that, because one gene treatment chip is used to perform all of the steps required for the gene treatment, the size of the gene treatment chip is increased, thus resulting in a larger size and a higher cost of the analysis apparatus. When analysis is performed plural times by using the same sample and the same analysis apparatus in order to increase the analysis accuracy, a long time is taken because of the necessity of performing all of the treatment steps repeatedly, and a cost is also increased because of using a plurality of expensive gene treatment chips. For those reasons, it is practically difficult to increase the analysis accuracy by performing the analysis plural times.

An object of the present invention is to provide a bio-analysis chip, a bio-analysis system, and a bio-analysis method, which are convenient in use and can promptly analyze microbes with high accuracy.

Another object of the present invention is to provide a bio-analysis method, which can promptly analyze microbes with reliable safety and high accuracy while using smaller and more inexpensive equipment.

To achieve the above objects, according to a first aspect, the present invention provides a bio-analysis chip comprising a collection member for collecting microbes in the atmosphere with the microbes adhering to the collection member, and a substrate in the form of a thin plate on which the collection member is mounted, wherein the substrate has a collection member receiving section in which the collection member is received, and a plurality of reagent reservoirs in which reagents for treating and analyzing the microbes are stored, the plurality of reagent reservoirs being connected to the collection member receiving section.

Preferred examples of the present invention according to the first aspect are as follows.
(1) The collection member receiving section is in the form of a shallow recess having an opening through which the atmosphere containing the microbes is introduced, the collection member being received at a bottom of the recess opposite to the opening, and the opening is enclosed by a sealing material in a state of the microbes adhering to the collection member.
(2) In above (1), an upper part of the collection member receiving section is communicated with an air hole opened to the outside in a state of the reagents being fed from the reagent reservoirs to the collection member receiving section.
(3) The collection member receiving section is arranged in a central area of the substrate, and the plurality of reagent reservoirs are arranged in surrounding relation to the collection member receiving section.
(4) In above (3), the plurality of reagent reservoirs are in the form of elongate fine channels each having one end connected to the collection member receiving section and the other end connected to a chip port which serves as a junction port communicating with the outside.
(5) The collection member is made of agar with a concentration of 2-5%, to which are added alcohols with a concentration of 40-80%.
(6) The bio-analysis chip is used to analyze genes of germs forming spores, and the plurality of reagent reservoirs include a reagent reservoir for storing a germination accelerator, a reagent reservoir for storing a cell lysin, and a reagent reservoir for storing chaotropic ions.
(7) The substrate and the collection member are each made of a material capable of being incinerated.

Also, to achieve the above objects, according to a second aspect, the present invention provides a bio-analysis system comprising a collector, bio-analysis chips constituted as a collection chip and an analysis chip, and an analysis apparatus, wherein the collection chip comprises a collection member for collecting microbes in the atmosphere with the microbes adhering to the collection member, and a collection-chip substrate in the form of a thin plate on which the collection member is mounted; the collection-chip substrate has a collection member receiving section in which the collection member is received, and a plurality of reagent reservoirs in which reagents for treating and analyzing the microbes are stored; the collector causes the microbes in the atmosphere to collide against and adhere to the collection member when the collection chip is set in the collector; the analysis chip includes an analysis-chip substrate in the form of a thin plate; the analysis-chip substrate has a sample pool for storing a sample having been subjected to pretreatment on the collection chip, and a plurality of reagent reservoirs for storing reagents used to perform posttreatment for analysis of the microbes; and the analysis apparatus causes the pretreatment to be performed in the collection chip when the collection chip is set in the analysis apparatus, and causes the postreatment to be performed in the analysis chip when the analysis chip is set in the analysis apparatus.

Preferred examples of the present invention according to the second aspect are as follows.
(1) The collector and the analysis apparatus are combined into one unit.
(2) The collector comprises a nozzle for sucking the atmosphere, a collection chamber provided in the outlet side of the nozzle, a mechanism for detachably attaching the collection chip, and an exhaust portion through which air sucked into the collection chamber is exhausted to the side opposite to the nozzle, the nozzle having a hole with an inner diameter of 4-15 mm.
(3) The analysis chip is arranged in a vertical posture in the analysis apparatus.
(4) The plurality of reagent reservoirs in the collection chip have respective one ends connected to the collection member receiving section and respective other ends connected to a plurality of chip ports which serve as junction ports communicating with the outside, and the analysis apparatus includes an analysis-apparatus base plate having a plurality of channels connectable to the chip ports of the collection chip, and a fluid supply mechanism for selectively supplying a fluid from the plurality of channels in the analysis-apparatus base plate to the chip ports of the collection chip through valves.
(5) In above (4), the plurality of reagent reservoirs in the analysis chip are connected to the plurality of chip ports which serve as the junction ports communicating with the outside, the analysis-apparatus base plate has a plurality of channels connectable to the chip ports of the analysis chip, and the fluid supply mechanism selectively supplies the fluid from the plurality of channels in the analysis-apparatus base plate to the chip ports of the analysis chip through the valves.

Further, to achieve the above objects, according to a third aspect, the present invention provides a bio-analysis method comprising the steps of setting, in a collector, a bio-analysis chip including a collection member receiving section in which a collection member is received, and a plurality of reagent reservoirs in which reagents for treating and analyzing microbes are stored, and operating a blowing mechanism in the collector such that the microbes in the atmosphere are caused to collide against and adhere to the collection member; and setting the bio-analysis chip containing the adhered microbes in an analysis apparatus, and operating a fluid feeding mechanism in the analysis apparatus such that the reagents in the plurality of reagent reservoirs in the bio-analysis chip are successively supplied to the collection member, thereby treating and analyzing the microbes.

Still further, to achieve the above objects, according to a fourth aspect, the present invention provides a bio-analysis method comprising the steps of setting, in a collector, a first bio-analysis chip including a collection member receiving section in which a collection member is received, and a plurality of reagent reservoirs in which reagents for performing pretreatment for analysis of microbes are stored, and operating a blowing mechanism in the collector such that the microbes in the atmosphere are caused to collide against and adhere to the collection member; setting the first bio-analysis chip containing the adhered microbes in an analysis apparatus, and operating a fluid feeding mechanism in the analysis apparatus such that the reagents in the plurality of reagent reservoirs in the first bio-analysis chip are successively supplied to the collection member, thereby performing the pretreatment for the analysis of the microbes; and setting, in the analysis apparatus, a second bio-analysis chip including a sample pool, a plurality of reagent reservoirs in which reagents for performing posttreatment for analysis of the microbes are stored, and a reaction cell, in a state of the sample pool being filled with the sample having been subjected to the pretreatment for the analysis, and operating the fluid feeding mechanism in the analysis apparatus such that the sample in the sample pool and the reagents in the plurality of reagent reservoirs are successively supplied to the reaction cell, thereby performing the posttreatment for the analysis of the microbes.

A preferred example of the present invention according to the fourth aspect is as follows.
(1) The bio-analysis method is used to analyze genes of germs forming spores, the pretreatment performed in the first bio-analysis chip comprises the steps of applying a germination accelerator and bringing cell walls into lysis, and the posttreatment performed in the second bio-analysis chip comprises the steps of binding the genes after the lysis of the cell walls to a gene binding carrier, washing a bound substance of the genes and the gene binding carrier, eluting the genes from the gene binding carrier, and detecting the eluted genes.

According to the present invention, the bio-analysis chip, the bio-analysis system, and the bio-analysis method can be obtained which are convenient in use and can promptly analyze microbes with high accuracy.

Also, according to the present invention, the bio-analysis method can be obtained which can promptly analyze microbes with reliable safety and high accuracy while using smaller and more inexpensive equipment.

### BRIEF DESCRIPTION OF THE DRAWINGS

FIG. 1 is a flowchart showing steps of a germ analysis method according to a first embodiment of the present invention;

FIG. 2 illustrates the configuration of a germ analysis system according to the first embodiment;

FIG. 3 is a perspective view showing the interior of a collector according to the first embodiment in a seeing-through way;

FIGS. 4A and 4B are perspective views showing a manner of mounting a collection chip to the collector shown in FIG. 3;

FIG. 5 is a front view of the collection chip according to the first embodiment;

FIG. 6 is a vertical sectional view of the collection chip shown in FIG. 5;

FIG. 7 is a front view of an analysis chip according to the first embodiment;

FIG. 8 is a sectional view taken along the line A-A′ in FIG. 7;

FIG. 9 is an enlarged view of a portion below a reaction cell of the analysis chip shown in FIG. 7;

FIG. 10 is an enlarged view showing a dam in a gene extraction area of the analysis chip shown in FIG. 7;

FIG. 11 is a perspective view showing the interior of an analysis apparatus according to the first embodiment in a seeing-through way;

FIG. 12 is a schemaic view, partly sectioned, showing the construction of the analysis apparatus shown in FIG. 11;

FIG. 13 is a front view showing a base plate in the analysis apparatus shown in FIG. 11; and

FIGS. 14A-14F show profiles in fluid handling according to the first embodiment.

### DESCRIPTION OF THE PREFERRED EMBODIMENTS

Several embodiments of the present invention will be described below. It is to be noted that the present invention is not limited to the embodiments disclosed below and can be modified in various ways in view of the related art, etc.

### First Embodiment

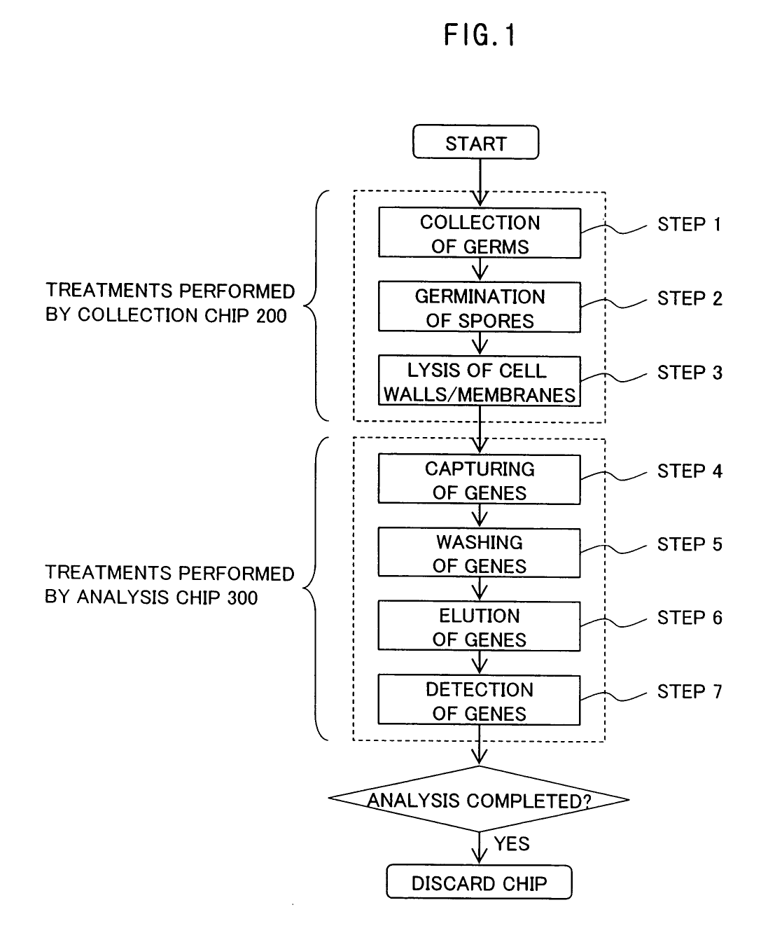

A first embodiment of the present invention will be described below with reference to FIGS. 1-14. The following description of the first embodiment is made in connection with an example in which germs forming spores are collected from the atmosphere, genes are extracted from the germs after treating the spores, and the extracted genes are amplified by the polymerase chain reaction, to thereby detect whether the objective germ as an analysis target is present. Examples of the germs forming the spores include bacillus and clostridium.

(Flow of Germ Analysis)

A germ analysis method according to the first embodiment mainly comprises the steps of collecting germs, adding a germination accelerator to germ spores for germination of the germ spores, extracting genes from the germinated germs, and amplifying and detecting the genes. Herein, the genes are extracted by the generally known solid-phase extraction method. The term “solid-phase extraction method” means a method of causing genes to be specifically bound to a solid surface, and eluting only the genes into an aqueous solution separately from other substances, thereby extracting the genes.

The germ analysis method according to the first embodiment will be described below with reference to FIG. 1. FIG. 1 is a flowchart showing steps of the germ analysis method according to the first embodiment of the present invention.

In step 1, germs are collected by a collision method. More specifically, the collision method used herein comprises the steps of sucking air as an analysis target through a nozzle inlet, ejecting the sucked air from a nozzle outlet at high speed, and collecting germs on a collection material in the form of a collision plate disposed in the nozzle outlet side. The germs contained in the sucked air are each given with an inertial force that is proportional to the square of a particle diameter, whereby the germs adhere to the collision plate. Such a collection method is advantageous in that the germs are collected in a concentrated state without making a filter clogged as experienced with a filter method.

In step 2, germ spores are germinated. More specifically, a germination accelerator is added to the spores of the collected germs (sample), and after the lapse of a predetermined time, the germ spores starts germination. In the germination stage, the germs destroy their own spores. With the germination, therefore, cell walls of the germs are brought into an exposed state.

In step 3, lysis of the cell walls and membranes is performed. Each germ has a double structure made up of a cell wall and a cell membrane. First, a particular enzyme is added to the sample to destroy the cell walls of the germs. Then, a solution containing chaotropic ions (negative ions each having a valence of −1 and a large molecule diameter) is mixed in the sample for lysis of the cell membranes of the germs by the action of the chaotropic ions. Simultaneously, the chaotropic ions denature many proteins contained in the sample and impede the action of nuclease (enzymes acting to decompose nucleic acids).

In step 4, genes are captured. More specifically, silica is added to the mixture obtained after the lysis in the above step. With the addition of silica, each gene and the silica are specifically bound to each other by the action of the chaotropic ion, thereby forming a gene-silica bound substance. In general, the gene and the silica are bound to each other by a method of passing the mixture after the lysis through a glass filter.

In step 5, the gene-silica bound substance is washed. If the proteins and the chaotropic ions contained in the sample are mixed in the extracted gene, detection of the gene with gene amplification is impeded. Therefore, an operation for washing the gene-silica bound substance is required. Usually, the gene-silica bound substance is washed by ethanol having a high concentration. Because a gene is hard to dissolve in such a liquid from its own specific nature, the gene adsorbed onto the silica is not eluted in the washing step.

In step 6, the gene is eluted from the silica. More specifically, after the washing, water or a solution having a low salt concentration is added to the gene-silica bound substance, thus causing the gene to elute from the silica.

In step 7, the eluted gene is detected. More specifically, reagents, such as a primer (single-chain DNA having the same base sequence as that of about 20 bases at both terminal ends of a target DNA area), DNA synthesis enzymes (polymerase), and 4 kinds of substrates (dNTP), are added to the eluted gene and a temperature cycle of “heat denaturation—annealing—synthesis of complementary strands” is applied to the gene, thereby amplifying the gene (polymerase chain reaction). The amplification of the gene can be detected in real time by injecting a fluorescent dye together with the above-mentioned reagents in advance and applying the temperature cycle while excitation light is irradiated.

(Configuration and Operation of Germ Analysis System)

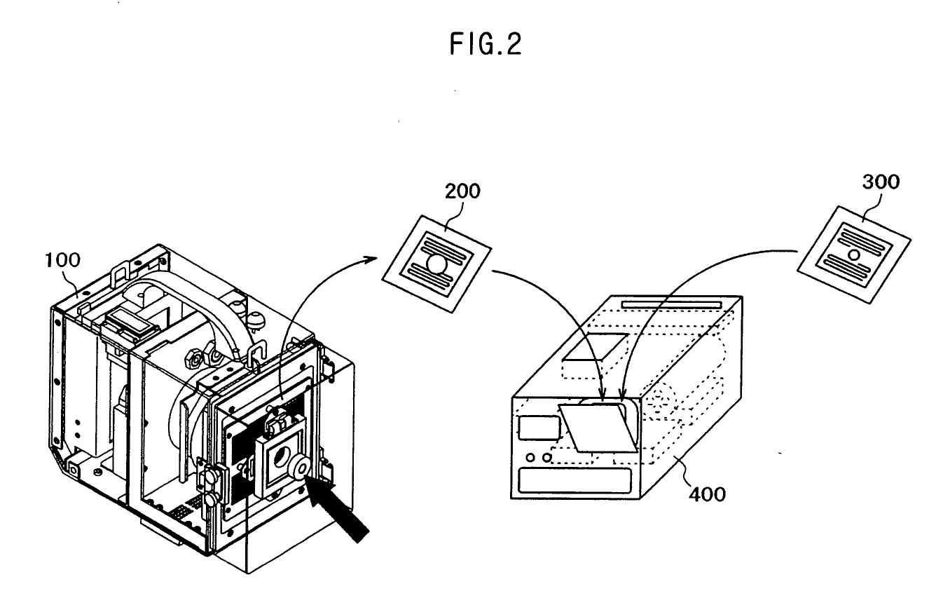

The configuration and operation of a germ analysis system according to the first embodiment will be described below with reference to FIG. 2. FIG. 2 illustrates the configuration of the germ analysis system according to the first embodiment of the present invention.

The germ analysis system comprises a collector 100, a microbe detection chip (200 and 300), and an analysis apparatus 400. The microbe detection chip is constituted as a first chip, i.e., a collection chip 200, and a second chip, i.e., an analysis chip 300.

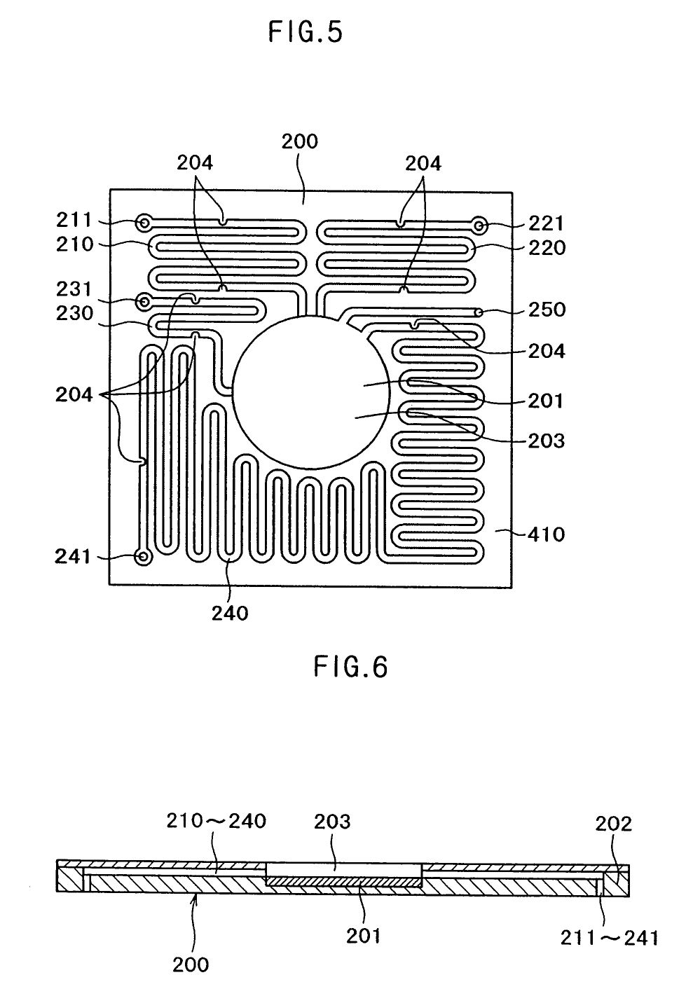

The collection chip 200 stores in advance a plurality of reagents used in the treatments from the step 2 (germination of spores) to the step 3 (lysis of cell membranes) in FIG. 1 and is set in the collector 100. Germs floating in the atmosphere are sucked into the collector 100 and are collected on a collection member 201 (see FIGS. 5 and 6) of the collection chip 200 set in the collector 100. Subsequently, the collection chip 200 is set in the analysis apparatus 400 after taking the collection chip 200 out of the collector 100 and closing an opening of a collection member receiving section 203 (see FIGS. 5 and 6) of the collection chip 200 by a sealing material, or after closing the opening of the collection member receiving section 203 of the collection chip 200 by the sealing material and taking the collection chip 200 out of the collector 100. In other words, the collection chip 200 is set in the analysis apparatus 400 in such a state that the opening of the collection chip 200 is closed by the sealing material with the microbes adhering to the collection member 201. By thus handling the collection chip 200 while the opening of the collection chip 200 is closed by the sealing material, the collection chip 200 can be handled with safety. The analysis apparatus 400 includes a liquid feeding mechanism for feeding the reagents in the collection chip 200 in a state where the collection chip 200 is set in the analysis apparatus 400, thereby carrying out the treatments to germinate the germ spores and to bring the cell membranes into lysis inside the collection chip 200.

As described above, since germs are collected by using the collection chip 200 storing a plurality of reagents in advance and the collection chip 200 including the collected germs is used as it is to start subsequent plural kinds of microbe analysis treatments, transition from the germ collection to the microbe analysis treatment can be realized with good convenience.

Thereafter, the collection chip 200 is taken out of the analysis apparatus 400, and a part of a liquid (in which the germs are already brought into lysis and which has no risk of contamination even if an inspection operator touches the liquid) obtained through the treatments in the collection chip 200 is transferred to the analysis chip 300.

The analysis chip 300 is then set in the analysis apparatus 400. At this time, the analysis chip 300 is set in the same place as that where the collection chip 200 has been set. The analysis chip 300 stores in advance a plurality of reagents used in the treatments from the step 4 (capturing of genes) to the step 7 (detection of genes) in FIG. 1. In a state of the analysis chip 300 being set in the analysis apparatus 400, the reagents in the analysis chip 300 are fed by using the liquid feeding mechanism in the analysis apparatus 400 to carry out the treatments from the gene extraction to the gene detection inside the analysis chip 300. After the completion of the analysis, the analysis chip 300 is taken out of the analysis apparatus 400 and is discarded.

As described above, since two types of the microbe analysis chips (i.e., the collection chip 200 and the analysis chip 300) store in advance all the reagents necessary in the steps from the germ pretreatment to the gene detection, it is possible to omit the complicated reagent dispensing operations which have been required in the past. More specifically, the known analysis process is complicated and has a risk of infection by the inspection operator because a sample containing germs is moved through many vessels during the analysis process such that a certain reagent is added to the sample for one treatment and is then transferred to another vessel for another treatment, and such an operation is repeated until all of the required treatments are completed. In contrast, according to the first embodiment, except for the step of transferring the sample between the two types of chips 200 and 300, the sample is kept from coming out of the chips and the analysis is performed in an enclosed system. Therefore, very high safety is ensured. Also, wastes to be discarded are only the chips 200 and 300. By using the chips 200 and 300 made of a material capable of being incinerated, a risk of secondary contamination can be reduced. Further, since the reagents stored in the two types of chips 200 and 300 are just for one kind of inspection, the chips 200 and 300 can be discarded after the one kind of inspection, thus enabling a germ inspection to be simply performed outdoor at a gene level with high accuracy.

In addition, since the two types of chips 200 and 300 storing the reagents are divided into the collection chip 200 for carrying out the treatments from the collection to the lysis of germs and the analysis chip 300 for carrying out the subsequent analysis treatments, the analysis can be easily performed plural times by using a sample that is subjected to the same treatments from the collection to the lysis of germs. As a result, analysis accuracy can be easily improved while ensuring safety.

(Construction and Operation of Collector)

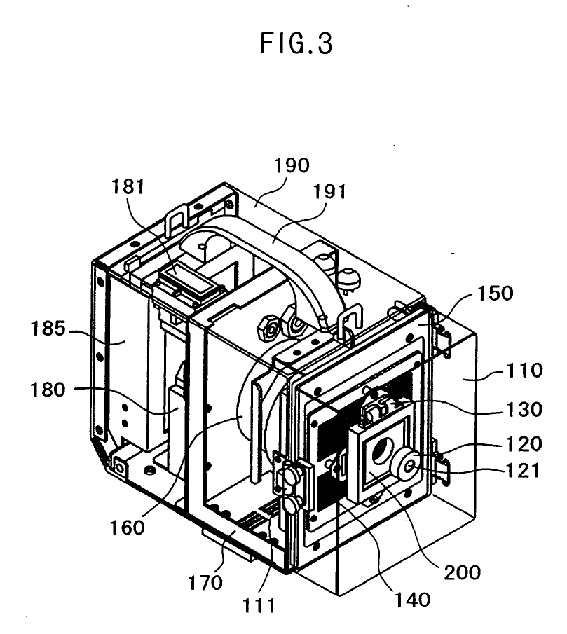

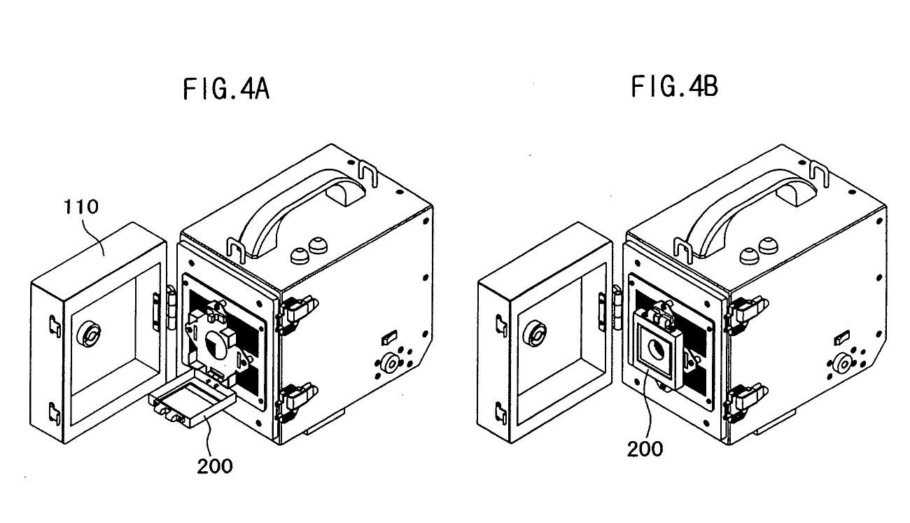

The construction and operation of the collector 100 will be described below in detail with reference to FIGS. 3 and 4. FIG. 3 is a perspective view showing the interior of the collector 100 according to the first embodiment of the present invention in a seeing-through way, and FIGS. 4A and 4B are perspective views showing a manner of mounting the collection chip 200 to the collector 100 shown in FIG. 3. Here, FIG. 4A shows a state where a lid 110 and a chip support 130 of the collector 100 are opened, and FIG. 4B shows a state where the collection chip 200 is mounted and the chip support 130 is closed from the state of FIG. 4A.

The collector 100 comprises, as shown in FIG. 3, the lid 110, a nozzle 120, a primary filter 121, the chip support 130, a secondary filter 140, a support plate 150, a fan motor 160, an exhaust port 170, a control unit 180, a display unit 181, a battery 185, and a casing 190.

The lid 110 is formed of a square or rectangular member provided with the nozzle 120, and it has fasteners 111 at both sides thereof. An inner diameter W of the nozzle 120 is closely related to the germ collection efficiency. As the inner diameter of the nozzle 120 decreases from 10 [mm], germs can be collected in a more concentrated state, but the speed of an airflow passing through the nozzle 120 is increased and so is a pressure loss. The pressure loss is increased in proportion to the square of the airflow speed. This results in that the load of the fan motor 160 is increased and the voltage of the battery 185 is lowered. If the inner diameter of the nozzle 120 is set to, e.g., 3 [mm] or below, the work load exceeds a level adaptable by the battery 185 (lithium-hydrogen) that can be mounted in the portable germ collector 100. For that reason, the inner diameter W of the nozzle 120 is preferably set to the range of 4-15 [mm]. More preferably, the inner diameter W of the nozzle 120 is set to the range of 8-12 [mm]. By so setting the nozzle inner diameter, germs can be collected in a region just under (or downstream of) the nozzle 120 in a concentrated state while obtaining high collection efficiency.

The primary filter 121 is mounted to the nozzle 120. The primary filter 121 is disposed to trap coarse particles in the atmosphere. To that end, the mesh size of the primary filter 121 is preferably in the range of 100-200 μm. In the season where the amount of pollens scattered increases, the mesh size of the primary filter 121 is preferably in the range of 10-100 μm. By so setting the mesh size of the primary filter 121, pollens having particle sizes of not smaller than 10 μm can be easily separated from germs having particle sizes smaller than 10 μm. Preferably, the primary filter 121 is detachable from the lid 110 and is made of stainless steel or a fluorocarbon resin so that the primary filter 121 can be easily washed and sterilized at high temperatures.

The chip support 130 is disposed above (or in front of) the secondary filter 140. As shown in FIGS. 4A and 4B, the chip support 130 can be opened and closed in such a manner that the collection chip 200 can be easily set in the collector 100 by closing the chip support 130 with the collection chip 200 held within the chip support 130. The chip support 130 serves as an outer peripheral frame for the collection chip 200 and is therefore contaminated with germs. For that reason, the chip support 130 is preferably detachable from the secondary filter 140 and is made of stainless steel or a fluorocarbon resin so that the chip support 130 can be easily washed and sterilized at high temperatures.

The secondary filter 140 is mounted to the support plate 150 and has the function of preventing fine particles, such as germs, which have not been trapped and collected by the collection chip 200, from being released to the atmosphere through the exhaust port 170. Preferably, a HEPA (High Efficiency Particulate Air) filter capable of trapping fine particles of not smaller than 0.3 μm at 99.97% or more is used as the secondary filter 140. More preferably, a ULPA (Ultra Low Penetration Air) filter capable of trapping fine particles of 0.1-0.2 μm at 99.999% or more is used. By using the ULPA filter, cleanness of the air released to the atmosphere through the exhaust port 170 can be further increased.

Inside the casing 190, the control unit 180, the display unit 181, and the battery 185 are disposed. A grip 191 is provided on an upper surface of the casing 190.

The operation of the collector 100 will be described below. When the fan motor 160 is driven, the atmosphere is sucked into the nozzle 120. The sucked air is accelerated in the nozzle 120 and passes through the primary filter 121. On that occasion, coarse particles in the sucked air are removed by the primary filter 121. Fine particles in the air sucked into the lid 110 collide against the collection member 201 disposed at the center of the collection chip 200 by their own inertia and adhere to the collection member 201. Then, the air sucked into the lid 110 passes through the secondary filter 140 and is exhausted to the outside through the exhaust port 170 formed below the fan motor 160. The secondary filter 140 removes the fine particles which have not been trapped by the collection chip 200.

(Construction and Operation of Collection Chip)

The construction and operation of the collection chip 200 will be described below in detail with reference to FIGS. 5 and 6. FIG. 5 is a front view of the collection chip 200 according to the first embodiment of the present invention, and FIG. 6 is a vertical sectional view of the collection chip 200 shown in FIG. 5.

The collection chip 200 is employed to perform the treatments from the step 1 (collection of germs) to the step 3 (lysis of cell membranes) in FIG. 1. More specifically, those treatments include the steps of setting the collection chip 200 in the collector 100 and collecting germs (step 1), the steps of removing the collection chip 200 from the collector 100, setting the collection chip 200 in the analysis apparatus 140, and germinating germ spores (step 2), and the step of bringing cell membranes into lysis (step 3).

The collection chip 200 is fabricated by using patterns, which are formed by the photolithography so as to define chip components. More specifically, the collection chip 200 is molded by transferring those patterns onto a resin. Most of the patterns constitute fine channels, and the patterns formed on the resin define the fine channels by bonding two resin plates to each other on which halves of the fine channels are formed. As a chip material, a resin being superior in disposability as wastes is more preferable than glass that requires a higher working cost and is apt to break. The kind of resin is not limited to particular one, but polydimethylsiloxane (PDMS: Silpot 184 made by Dow Corning Asia Co., Ltd.) having excellent characteristics given below was used in the first embodiment:

good adaptability with living bodies (usual silicone rubber is physiologically inactive)

mold transferability with high accuracy on order of submicrons (because of having low viscosity and high fluidity before hardening, it is able to permeate well into every corners of complicated patterns)

low cost (it is generally cheaper than Pyrex (registered trade name) glass that is a conventional versatile micro-device material)

it is easily disposable by incineration

The collection chip 200 comprises the collection member 201 for collecting microbes (germs forming spores in this embodiment) from the atmosphere with the microbes adhering to it, and a substrate 202 in the form of a thin plate on which the collection member 201 is mounted. The collection chip 200 has a collection member receiving section 203 for receiving the collection member 201, a plurality of reagent reservoirs 210, 220, 230 and 240, chip ports 211, 221, 231 and 241 opened in a chip rear surface, and an air hole 250 opened in a chip front surface.

The plurality of reagent reservoirs include a germination accelerator reservoir 210 for storing the germination accelerator, an enzyme A reservoir 220 and an enzyme B reservoir 230 for storing two kinds of cell lysins, and a chaotropic reservoir 240 for storing chaotropic ions. The plurality of reagent reservoirs 210-240 are arranged so as to surround the collection member receiving section 203. With such an arrangement, the substrate 202 can be made compact.

The reagent reservoirs 210-240 are each constituted as an elongate channel. Each of the reagent reservoirs 210-240 is preferably in the form of a narrow and long channel. The reason is as follows. In order to feed the reagents stored in the reagent reservoirs 210-240, gas (fluid) is supplied to the reagent reservoirs 210-240 from the side behind the reagent reservoirs 210-240. On that occasion, if the reagent reservoirs 210-240 are not in the form of narrow and long channels, the reagent is pushed out only through some channel that allows easier passage of the reagent, while the reagents fed through the other channels are left in the reagent reservoirs. Thus, the reagent reservoirs 210-240 in the form of narrow and long channels are effective in reducing the amounts of reagents consumed. A cross-sectional shape of each channel is not limited to a particular one, but a width/length ratio of the channel is preferably not larger than 10. If the width/length ratio is larger than 10, there is a risk that the resin forming a ceiling portion of the channel may flex and the rectangular shape of the channel may be deformed. The narrow and long channels of the reagent reservoirs 210-240 are formed in meandering patterns. Such an arrangement makes it possible to reduce an area occupied by the channels in the substrate 202 and to ensure that the necessary amounts of reagents are stored in the channels.

One ends of the reagent reservoirs 210-240 are communicated with the collection member receiving section 203, and the other ends of the reagent reservoirs 210-240 are communicated with the chip ports 211-241, respectively. Dams 204 are provided in each of the reagent reservoirs 210-240 at positions near the one end and the other end thereof. The provision of the dams 204 prevents the reagent stored in each of the reagent reservoirs 210-240 from flowing out unintentionally with higher reliability. The chip ports 211-214 serve as junctions with external channels. More specifically, the germination accelerator reservoir 210, the enzyme A reservoir 220, the enzyme B reservoir 230, and the chaotropic reservoir 240 are all communicated with the collection member receiving section 203. Therefore, the collection member receiving section 203 can be connected to the external channels through the germination accelerator reservoir 210, the enzyme A reservoir 220, the enzyme B reservoir 230, the chaotropic reservoir 240, and the chip ports 211-214. Inflow of air from the side communicating with the collection member receiving section 203 can be prevented by narrowing the channel width of each of the reagent reservoirs 210-240 to 10-50 μm.

Preferably, the germination accelerator reservoir 210 has a volume of 20-100 μL, the enzyme A reservoir 220 has a volume of 20-100 μL, the enzyme B reservoir 230 has a volume of 5-20 μL, and the chaotropic reservoir 240 has a volume of 400-800 μL. Lysis of the cell membranes is accelerated by setting the volume of the chaotropic ions to be twice or larger than the sum of the volume of the germination accelerator and the volume of the two kinds of cell lysins. Such a volume ratio is more preferably 4 times or larger and most preferably 8 times or larger.

A preferable material of the collection member 201 is agar. Agar is featured in having “adhesion” attributable to free water on the gel surface (i.e., water among gel meshes). The concentration of agar is preferably in the range of 2-5% and most preferably in the range of 3-4%. If the agar concentration is lower than 2%, the amount of water is too much and the strength of the collection member 201 against which air continuously collides at high speed is insufficient. On the other hand, if the agar concentration is higher than 6%, the amount of water on the agar surface (i.e., free water) is too less and the adhesion of the collection member 201 is significantly reduced.

Alcohols are preferably added to increase the strength of agar and to prevent evaporation of water. The added alcohols serve as an agent for preventing freezing and drying of the agar and increasing the gel strength. Practical examples of the alcohols include ethylene alcohol, isopropyl alcohol, 1,3-buthane diol, ethylene glycol, propylene glycol, and glycerin. The amount of alcohols added is preferably in the range of 40-80% of the agar and more preferably in the range of 50-70%. If the alcohol amount is less than 40%, water evaporation is not sufficiently prevented. On the other hand, if the alcohol amount exceeds 80%, the amount of water on the agar surface (i.e., free water) is too less and the adhesion of the collection member 201 is reduced.

One example of practical usage of the collection chip 200 will be described below in detail.

The collection chip 200 is attached to the chip support of the collector 100, and the atmosphere is sucked for a predetermined time. The amount of atmosphere sucked is, e.g., about 1000 L. Germs contained in the sucked atmosphere adhere to the surface of the collection member 201 in the collection chip 200. Then, the collection chip 200 is detached from the chip support and is set in the analysis apparatus 400 after closing the opening of the collection member receiving section 203 of the collection chip 200 by a sealing material. Although the sealing-off of the collection member receiving section 203 may be manually performed, an automatic sealing mechanism is preferably provided in the collector 100. With the sealing-off of the collection member receiving section 203, the germs are positively prevented from leaking to the outside of the collection chip 200 and higher safety is ensured.

The reagent reservoirs 210-240 of the collection chip 200 are connected to corresponding channels in the analysis apparatus 400 through the chip ports 211-241. Accordingly, the germination accelerator, one kind of cell lysin (enzyme A), the other kind of cell lysin (enzyme B), and the chaotropic ions, which are stored in the reagent reservoirs 210-240 in advance, can be fed into the collection member receiving section 203 (specifically, onto the collection member 201) at intervals of a predetermined time by supplying air from the channels in the analysis apparatus 400 through the chip ports 211-241 in accordance with predetermined control operations. Although the front surface (opening) of the collection member receiving section 203 is sealed off, the air hole 250 is communicated at one side with a part of the collection member receiving section 203 and is opened at the other side to the atmosphere. Therefore, when the reagents are fed, air existing above the collection member 201 (or inside the collection member receiving section 203) is released to the atmosphere through the air hole 250. In particular, because the air hole 250 is communicated with an upper part of the collection member receiving section 203, it is possible to reliably release the air in the collection member receiving section 203 when the reagents are fed.

The feeding process of the reagents from the reagent reservoirs 210-240 to the collection member 201 will be described below in more detail.

First, 100 μL of the germination accelerator is fed to the collection member 201. The germination accelerator is preferably bouillon containing alanine, adenosine, and glucose. Particularly, bouillon containing 1 mM-10 mM of L-alanine is optimum. After the lapse of 10 minutes or longer, germ spores start germination, and after the lapse of 30 minutes, 50% or more of all the germ spores start germination. For that reason, the spore germination step is preferably continued for 30 minutes or longer. A temperature condition for germinating the germ spores is preferably in the range of 35° C.-40° C. and most preferably in the range of 35° C. -37° C. In the germination stage of the germ spores, the germs destroy their own spores. With the germination, therefore, cell walls of the germs are brought into an exposed state.

Then, the two kinds of protein denaturing enzymes A and B for bringing the cell walls of the germs into lysis are successively fed to the collection member 201, followed by holding it at an optimum temperature for a predetermined time. The time of the enzyme treatment is preferably not shorter than 10 minutes and more preferably 30 minutes for each type of enzyme. As the protein denaturing enzymes, 100 μL of lysozyme (optimum temperature: 37° C.) and 20 μL of protease K (optimum temperature: 55° C.-60° C.) are preferably used. With those enzyme treatments, cell membranes of the germs in the collection chip 200 are brought into an exposed state. Note that while the germination accelerator and lysozyme may be injected at the same timing to perform the respective treatments, adding lysozyme and protease K at the same time is not preferable because the simultaneous addition of them reduces the enzyme activity.

Finally, 800 μL of the chaotropic ions are fed to the collection member 201. With the feeding of the chaotropic ions, the cell membranes of the germs are destroyed and germ genes are released to the outside of the cells. The chaotropic ions are given, for example, by guanidine thiocyanate, guanidine hydrochloride, sodium iodide, and potassium bromide. As one of usages of the collection chip 200, it is conceivable to maintain the reagent activity by keeping the collection chip 200 in cold or frozen storage. In view of such a usage, guanidine hydrochloride is preferable because composition change is very small when it is sealed in the collection chip 200 and kept in cold or frozen storage.

Preferably, the chaotropic salt contains a surfactant and/or a buffer. The surfactant is not limited to a particular one, and practical examples of the surfactant include Tween-20 and TritonX-100. Also, the buffer is not limited to a particular one, and practical examples of the buffer include tris-hydrochloride and potassium dihydrogenphosphate-sodium tetraborate.

Through the above-described steps, the spores and the cell walls of the germs collected in the collection chip 200 can be treated. In other words, the steps 2 and 3 in FIG. 1 can be automatically performed in the chip, and the operations for dispensing the reagents can be omitted. The collection chip 200 covers the steps ranging from the collection to the pretreatment of germs, and the analysis chip 300 covers the steps for analyzing germ genes. In order to perform the analysis for the same sample plural times or to set plural kinds of germs as analysis targets from the viewpoint of increasing the analysis accuracy, it is preferable to perform the gene analysis by dispensing the sample obtained through the treatments in one collection chip 200 to a plurality of analysis chips 300. For that reason, the two types of chips 200 and 300 are employed.

When the collection chip 200 is provided to a user in a frozen state and the user keeps the collection chip 200 in frozen storage at 0° C., the reagent activity is maintained for one month. Also, when the user keeps the collection chip 200 in frozen storage at −20° C., the reagent activity can be maintained for a half year or longer.

(Construction and Operation of Analysis Chip)

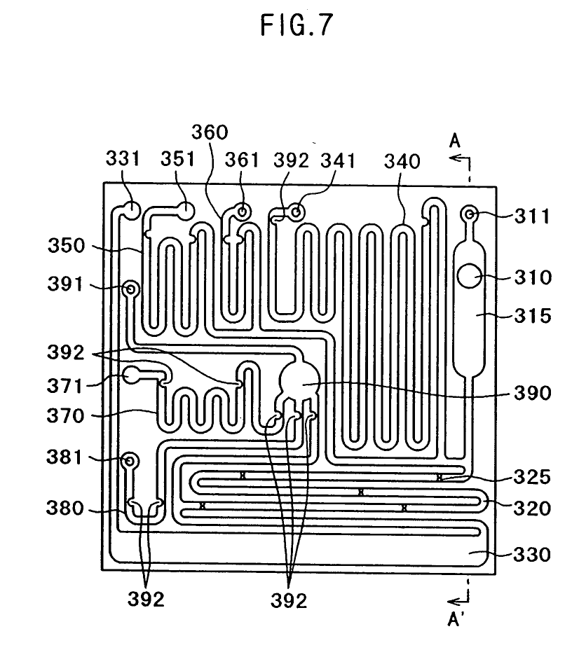

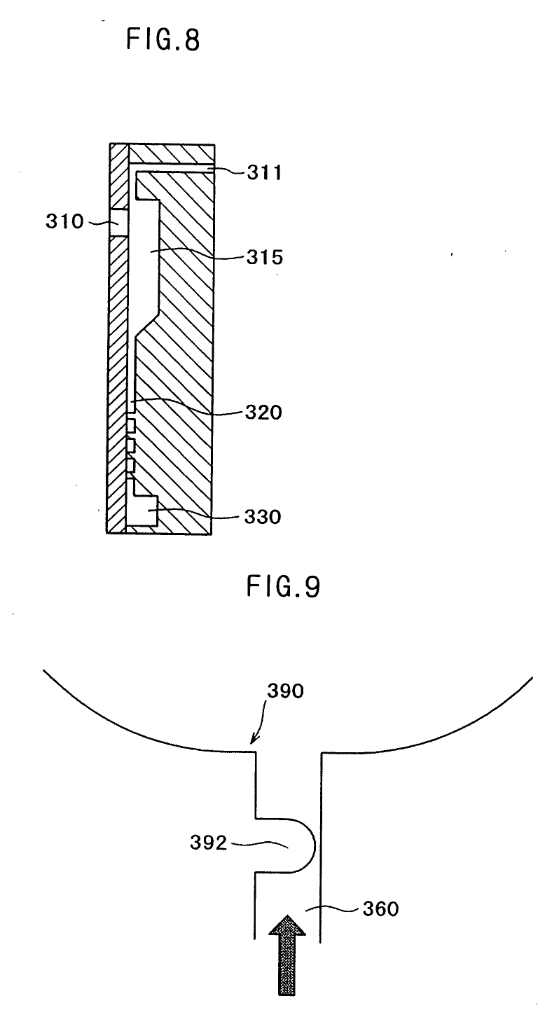

The analysis chip 300 will be described below in detail with reference to FIGS. 7-10. FIG. 7 is a front view of the analysis chip 300 according to the first embodiment of the present invention, and FIG. 8 is a sectional view taken along the line A-A′ in FIG. 7. Note that FIG. 8 is the sectional view when the analysis chip 300 is arranged in a vertical posture.

The analysis chip 300 is used to perform the treatments from the step 4 (capturing of genes) to the step 7 (detection of genes) in FIG. 1. The analysis chip 300 to which a part of the liquid obtained through the treatments performed in the collection chip 200 has been transferred from the collection chip 200 is set in the analysis apparatus 400. The analysis chip 300 stores in advance a plurality of reagents used in the treatments from the step 4 (capturing of genes) to the step 7 (detection of genes) in FIG. 1. In a state of the analysis chip 300 being set in the analysis apparatus 400, the reagents in the analysis chip 300 are fed by using the fluid feeding mechanism in the analysis apparatus 400 to carry out the treatments from the gene extraction to the gene detection inside the analysis chip 300. Additionally, the analysis chip 300 is made of the same material, i.e., the same resin, as that used to form the collection chip 200.

The analysis chip 300 has a sample inlet port 310 opened in a chip front surface, a sample pool 315, a gene extraction area 320 containing a gene binding carrier filled in its channel, a liquid waste cell 330, a washing liquid A reservoir 340 for storing a washing liquid A, a washing liquid B reservoir 350 for storing a washing liquid B, an eluent reservoir 360 for storing a gene eluent, a gene amplification reagent A reservoir 370 for storing a gene amplification reagent A, a gene amplification reagent B reservoir 380 for storing a gene amplification reagent B, a reaction cell 390 for amplifying and detecting genes, and chip ports 311, 331, 341, 351, 361, 371 and 381 opened in a chip rear surface. The reagent reservoirs 340-380 are each constituted as an elongate channel similarly to the reagent reservoirs 210-240 in the collection chip 200. A cross-sectional shape of each channel of the reagent reservoirs 340-380 is not limited to a particular one, but a width/length ratio of the channel is preferably not larger than 10 similarly to the channel in the collection chip 200. If the width/length ratio is larger than 10, there is a risk that the resin forming a ceiling portion of the channel may flex and the rectangular shape of the channel may be deformed. In this first embodiment, the channels of the reagent reservoirs 340-380 are each formed to have a length of 1 mm and a width of 1 mm.

The chip ports 311-381 are formed at respective one ends of the sample pool 315, the liquid waste cell 330, and the reagent reservoirs 340-380. The chip ports 311-381 serve as junctions with the outside, i.e., the channels in the analysis apparatus 400. In order to feed the reagents stored in the reagent reservoirs 340-380, air is supplied to the reagent reservoirs 340-380 from the outside of the analysis chip 300, i.e., from the analysis apparatus 400, through the chip ports 341-381. Instead of air, water or another fluid (e.g., nitrogen gas) causing no interference with the analysis may also be used.

The sample pool 315, the washing liquid A reservoir 340, the washing liquid B reservoir 350, and the eluent reservoir 360 are all communicated with the gene extraction area 320. Also, the gene extraction area 320, the gene amplification reagent A reservoir 370, and the gene amplification reagent B reservoir 380 are all communicated with the reaction cell 390. FIG. 9 is an enlarged view of a portion below the reaction cell 390. As shown, dams 392 are provided in the form of partial projections to narrow channels extending from the reaction cell 390 so that the gene amplification reagent A fed from the gene amplification reagent A reservoir 370 to the reaction cell 390 and the gene amplification reagent B fed from the gene amplification reagent B reservoir 380 to the reaction cell 390 are prevented from flowing backward once those reagents have entered the reaction cell 390. The channel size is preferably narrowed to 1/10- 1/20.

Preferably, the sample pool 315 has a volume of 100-200 μL, the gene extraction area 320 has a volume of 100-200 μL, the washing liquid A reservoir 340 has a volume of 80-200 μL, the washing liquid B reservoir 350 has a volume of 20-50 μL, the eluent reservoir 360 has a volume of 10-20 μL, the gene amplification reagent A reservoir 370 has a volume of 10-20 μL, and the gene amplification reagent B reservoir 380 has a volume of 20-30 μL.

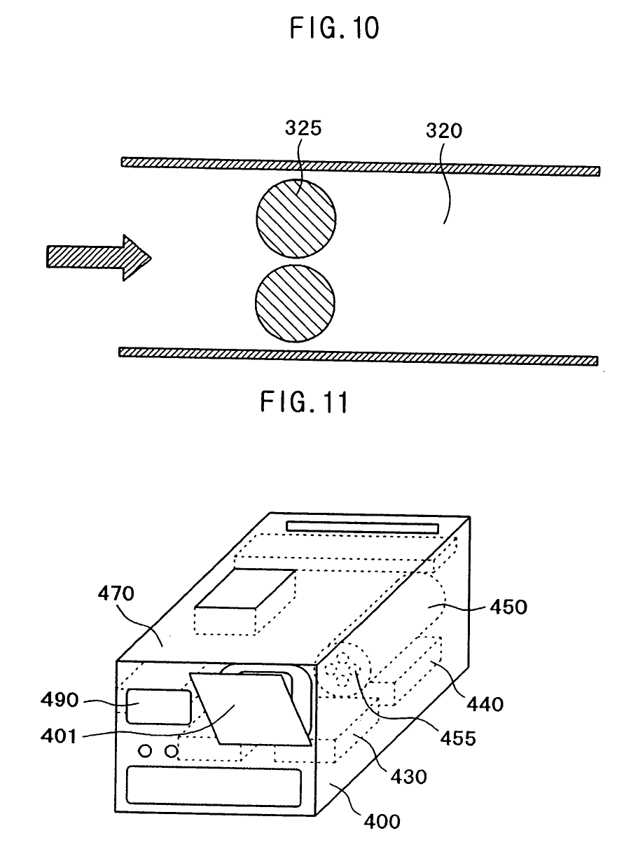

Quartz wool, glass wool, glass fibers, and glass beads can be employed as the gene binding carrier filled in the gene extraction area 320. When glass beads are employed, the bead size is preferably not larger than 50 μm to increase a contact area. In consideration of the necessity of preventing the beads from flowing out of a storage channel, the optimum bead size is in the range of 20-30 μm. To prevent unintentional outflow of the gene binding carrier, a dam 325 is preferably provided at one or more positions in the channel constituting the gene extraction area 320. FIG. 10 shows one example of the construction of the dam 325. By narrowing the channel size to 10-50 μm at several positions in the channel of the gene extraction area 320, it is possible to dam the gene binding carrier from flowing out of the channel by the narrowed channel. If the channel size is smaller than 10 μm, the fluid resistance is so increased as to cause a difficulty in fluid control. Therefore, the channel size defined by the provision of the dam 325 is preferably in the range of 10-50 μm.

(Construction and Operation of Analysis Apparatus)

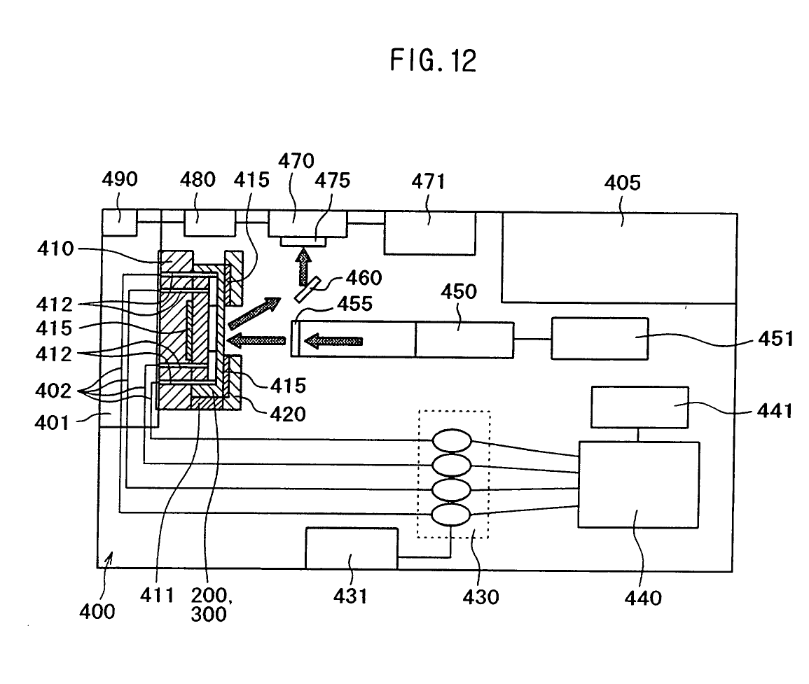

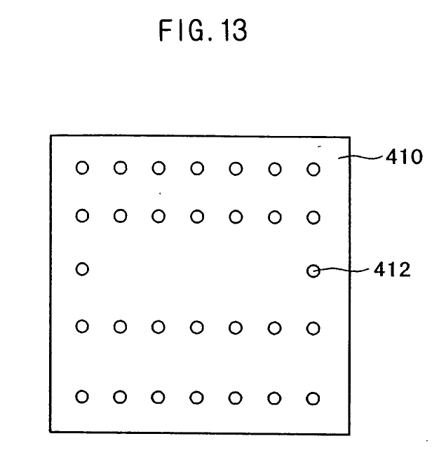

The construction and operation of the analysis apparatus 400 will be described below in detail with reference to FIGS. 11-13. FIG. 11 is a perspective view showing the interior of the analysis apparatus 400 according to the first embodiment of the present invention in a seeing-through way, FIG. 12 is a schematic view, partly sectioned, showing the construction of the analysis apparatus 400 shown in FIG. 11, and FIG. 13 is a front view showing a base plate 410 in the analysis apparatus 400 shown in FIG. 11.

The analysis apparatus 400 mainly comprises four components, i.e., a chip mounting unit, a fluid system, a temperature control system, and an optical detection system.

The collection chip 200 or the analysis chip 300 is set on the base plate 410 disposed inward of a front lid 401. In the first embodiment, since the chip 200 or 300 is set in a vertical posture, a chip stopper 411 for stopping and supporting the chip 200 or 300 is disposed under the base plate 410. When the chip 200 or 300 is set on the base plate 410 and the front lid 401 is closed, the chip 200 or 300 is fixedly held between the base plate 410 and a chip holder 420. A temperature control mechanism 415 for keeping the temperature of the chip 200 or 300 at an optimum level is incorporated in each of the base plate 410 and the chip holder 420. Although various types of heating elements can be used as the temperature control mechanism 415, one preferable example is a Peltier element. In the case of using a Peltier element, heating and cooling operations for the chip 200 or 300 can be simply controlled just by changing the direction of an applied current.

A plurality of base-plate channels 412 are formed in the base plate 410 on which is set the chip 200 or 300. The base-plate channels 412 have one ends communicated with the chip ports 211-241 or 311-381 of the chip 200 or 300, and have the other ends communicated with a plurality of channels 402 formed inside the analysis apparatus 400. Because the plurality of base-plate channels 412 are formed in the base plate 410 in advance, the base plate 410 is adaptable for the chip ports 211-241 or 311-381 of any of the collection chip 200 and the analysis chip 300. Thus, the analysis apparatus 400 can offer a platform usable in performing the treatments for the collection chip 200 and the analysis chip 300.

The plurality of apparatus channels 402 are connected to a pump 440 through respective valves 430. In order to feed the reagent in some reagent reservoir of the chip 200 or 300, the corresponding valve 430 is changed over such that a fluid (air or another gas) is supplied to only the channel communicating with the relevant reagent reservoir. Stated another way, the fluid delivered from the pump 440 reaches the interior of the chip 200 or 300 through the selected apparatus channel 402 and the selected base-plate channel 412, whereby the reagent in the corresponding reagent reservoir is fed as desired. Because the reagent is stored just in a predetermined amount in the reagent reservoir beforehand, the feeding operation can be performed simply by discharging all the reagent in the reagent reservoir under time control, and the pump 440 is not required to have high accuracy in feeding the fluid. Thus, the pump 440 can be constituted by a simple and small-sized one with the function of only blowing, not including suction.

As described above, the valves 430 for controlling fluid flows are preferably disposed not within the chip, but within the analysis apparatus 400. With such an arrangement, the chip contains no mechanical parts and can be realized as a small-sized and disposable chip.

The optical detection system comprises a light source 450 for illuminating excitation light to the genes in the chip reaction cell 390, an excitation filter 455 allowing passage of only a particular wavelength of the excitation light, a mirror 460 for changing an optical path of fluorescence generated from the chip reaction cell 390, a detection filter 475 allowing passage of only a particular wavelength of the fluorescence, and a photodetector 470 for measuring the fluorescence. While the light source 450 can be selected from among light sources covering various wavelength ranges, a xenon lamp having a wide wavelength range can be used as one example. When the wavelength used is limited, an LED emitting a particular wavelength may be used. Any of a CCD camera, a photomultiplier, and a photodiode can be used as the photodetector 470. However, the use of a photodiode is preferable from the viewpoint of downsizing the analysis apparatus 400. An optical signal corresponding to the target gene and detected by the photodetector 470 is converted to a digital signal by an optical signal converter 480, and the signal intensity is displayed on a data display screen 490.

In addition, the analysis apparatus 400 includes control mechanisms for controlling the various components. More specifically, the analysis apparatus 400 includes a valve control mechanism 431 for controlling the valves 430, a pump control mechanism 441 for controlling the pump 440, a light source control mechanism 451 for controlling the light source 450, and a photodetector control mechanism 471 for controlling the photodetector 470.

According to the first embodiment, the analysis apparatus 400 can be provided as a small-sized and portable apparatus just in combination of the small analysis chip 300 including no mechanical parts and placed on the base plate 410, the photodetector 470 having a simple structure, and the other associated components.

(Analysis Procedures)

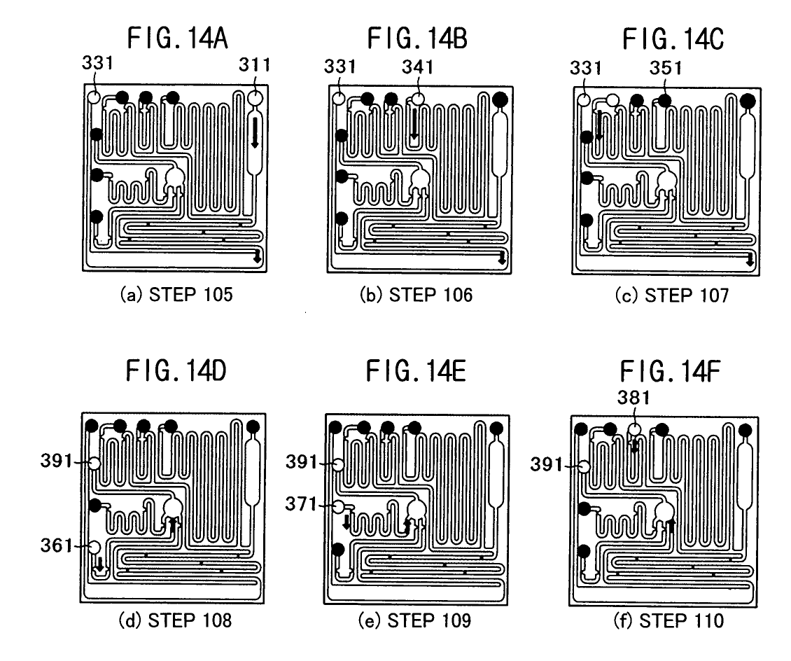

The procedures of analysis carried out by using the analysis chip 300 and the analysis apparatus 400 will be described below with reference to FIGS. 7, 12 and 14A-14F. FIGS. 14A-14F show profiles in fluid handling according to the first embodiment.

The analysis procedures using the analysis chip 300 are primarily performed as follows.

First, the germ sample obtained in the collection chip 200 by bringing the cell walls into lysis is transferred from the collection chip 200 to the analysis chip 300. In the analysis chip 300, the germ sample is supplied to the channel in which the gene binding carrier is filled. Further, the washing liquid for washing protein, etc. contained in the sample is fed to the channel in which the gene binding carrier is filled. Then, the eluent for eluting genes adsorbed on the gene binding carrier is also fed to the channel in which the gene binding carrier is filled. The eluted genes are fed to the reaction cell for the gene detection. The presence or absence of the objective gene as an analysis target is then detected.

One example of the analysis procedures will be described below in more detail. Note that steps 101-104 and 111-113 described below are not shown in the drawing.

Initially, the analysis chip 300 having been kept in cold or frozen storage is thawed at room temperature. The thawed analysis chip 300 contains the five kinds of reagents, i.e., the washing liquid A, the washing liquid B, the gene eluent, the gene amplification reagent A, and the gene amplification reagent B which are stored in the washing liquid A reservoir 340, the washing liquid B reservoir 350, the eluent reservoir 360, the gene amplification reagent A reservoir 370, and the gene amplification reagent B reservoir 380, respectively. By providing, to the user, the analysis chip 300 in which the reagents required just for one inspection are stored in advance, the reagents are not wastefully consumed even when the analysis chip 300 is discarded after only one inspection, and therefore economy is improved. On the user side, since the operations for dispensing the reagents into the reagent reservoirs can be omitted, it is possible to not only cut an inspection time, but also to keep the user from contamination. Further, when the analysis chip 300 is provided to the user in a frozen state and the user keeps the analysis chip 300 in frozen storage at 0C, the reagent activity is maintained for one month. Also, when the user keeps the analysis chip 300 in frozen storage at −20° C., the reagent activity can be maintained for a half year or longer. Thus, by storing the reagents just for one inspection in the disposable analysis chip 300 in advance and providing the analysis chip 300 to the user in the cold or frozen state, simple analysis environments can be realized. (Step 101) After thawing the analysis chip 300, about 100 μL of the liquid obtained through the treatments of the collection chip 200 is transferred to the sample inlet port 310 of the analysis chip 300. (Step 102)

Then, a hole of the sample inlet port 310 is closed with a cover. The cover is preferably formed of a thin resin sheet made of the same material as that of the analysis chip 300. The thin resin sheet is advantageous in that good adhesion is obtained between two contact members made of the same resin and that it is inexpensive and suitable for being discarded after use. Although the sample inlet port 310 can be manually closed with the cover, a mechanism for automatically covering the sample inlet port 310 is preferably provided the analysis apparatus 400. (Step 103)

After opening the front lid 401 of the analysis apparatus 400, the analysis chip 300 is set in the analysis apparatus 400 by inserting it along chip guides provided on the front lid 401, and the front lid 401 of the analysis apparatus 400 is closed. Responsively, the analysis chip 300 is fixedly held on the base plate 410, and the chip ports of the analysis chip 300 are communicated with the apparatus channels 402. While the analysis chip 300 can be arranged in any of horizontal and vertical postures, the following description is made of the case of the analysis chip 300 being arranged in the vertical posture. (Step 104)

Then, the valves 430 in the analysis apparatus 400 are selectively changed over such that the fluid is supplied from the pump 440 to only the sample port 311 (ports 311, 331: opened, other ports: closed, a white circle in FIG. 14 represents the valve being open and a black circle represents the valve being closed). The fluid used here can be selected from gases, such as air and nitrogen, which do not impair the reagent activity when brought into contact with the reagents. The sample in the sample pool 315 is moved to the gene extraction area 320. The germ genes in the sample are bound to the gene binding carrier filled in the gene extraction area 320 by the action of the chaotropic ions in the sample. In order to promote the binding between the germ genes and the gene binding carrier, a time taken by the sample to pass through the gene extraction area 320 is preferably set not shorter than 10 minutes. The sample having passed through the gene extraction area 320 is gathered in the liquid waste cell 330. The fluid (gas) used for feeding the sample is drained to the liquid waste port 331. With the analysis chip 300 arranged in the vertical posture, the sample can be prevented from leaking through the liquid waste port 331. (Step 105)

Then, the valves 430 in the analysis apparatus 400 are selectively changed over such that the sample port 311 is closed and the washing liquid A port 341 is opened. The fluid is thereby supplied from the pump 440 to only the washing liquid A port 341 (ports 331, 341: opened, other ports: closed). With the supply of the fluid, 200 μL of the washing liquid A in the washing liquid A reservoir 340 is fed to the gene extraction area 320. The washing liquid A preferably contains chaotropic ions given, for example, by guanidine thiocyanate, guanidine hydrochloride, sodium iodide, and potassium bromide. Protein remaining in the gene extraction area 320 is removed by the washing liquid A. The washing liquid A having passed through the gene extraction area 320 is gathered in the liquid waste cell 330. (Step 106)

Then, the valves 430 in the analysis apparatus 400 are selectively changed over such that the washing liquid A port 341 is closed and the washing liquid B port 351 is opened. The fluid is thereby supplied from the pump 440 to only the washing liquid B port 351 (ports 331, 351: opened, other ports: closed). With the supply of the fluid, 50 μL of the washing liquid B in the washing liquid B reservoir 350 is fed to the gene extraction area 320. The washing liquid B is preferably ethanol having a high concentration of not less than 50% or a potassium acetate solution. The chaotropic ions remaining in the gene extraction area 320 are removed by the washing liquid B. The washing liquid B having passed through the gene extraction area 320 is gathered in the liquid waste cell 330. (Step 107)

Then, the valves 430 in the analysis apparatus 400 are selectively changed over such that the liquid waste port 331 and the washing liquid B port 351 are closed and the gene amplification reagent A port 361 and the reaction cell port 391 are opened. The fluid is thereby supplied from the pump 440 to only the gene amplification reagent A port 361 (ports 361, 391: opened, other ports: closed). With the supply of the fluid, 10 μL of the gene amplification reagent A in the gene amplification reagent A reservoir 370 is fed to the reaction cell 390. The gene amplification reagent A is made up of four kinds of dNTP (dATP, dCTP, dGTP and dTTP), a buffer (e.g., TRIS-HCl, KCl or MgCl₂), a primer, etc. The fluid (gas) used for feeding the sample is drained to the reaction cell port 391. (Step 108)

Then, the valves 430 in the analysis apparatus 400 are selectively changed over such that the gene amplification reagent A port 361 is closed and the gene amplification reagent B port 371 is opened. The fluid is thereby supplied from the pump 440 to only the gene amplification reagent B port 371 (ports 371, 391: opened, other ports: closed). With the supply of the fluid, 30 μL of the gene amplification reagent B in the gene amplification reagent B reservoir 380 is fed to the reaction cell 390. The gene amplification reagent B is made up of a DNA synthesis enzyme (such as Taq DNA polymerase, Tth DNA polymerase, Vent DNA polymerase, or thermosequenase), and a fluorescent dye (such as ethidium bromide, SYBR GREEN (made by Molecular Probe, FAM, or ROX). (Step 109)

Then, the valves 430 in the analysis apparatus 400 are selectively changed over such that the gene amplification reagent B port 371 is closed and the eluent port 381 is opened. The fluid is thereby supplied from the pump 440 to only the eluent port 381 (ports 381, 391: opened, other ports: closed). With the supply of the fluid, 10 μL of the eluent in the eluent reservoir 360 is fed to the gene extraction area 320. Sterilized distilled water or a buffer solution, e.g., TRIS-EDTA or TRIS-acetate, can be used as the eluent. The eluent serves to elute the genes captured by the gene binding carrier in the gene extraction area 320. The eluted genes are fed to the reaction cell 390. (Step 110)

Through the above-described procedures, the germ genes and the two kinds of the gene amplification reagents are introduced to the reaction cell 390 of the analysis chip 300. In order to amplify and detect the germ genes in the reaction cell 390, the temperature control mechanism 415 is operated to apply a temperature cycle such that the temperature of the reaction cell 390 rises and lowers between two setting values, mentioned below, in a repeated way. (Step 111)

The temperature cycle is performed, by way of example, as follows:
“10-30 seconds at 90-95° C.⇄10-30 seconds at 65-70° C.”×30-45 times

One preferred example of the temperature cycle is performed as follows:
“30 seconds at 94° C. ⇄30 seconds at 68° C.”×45 times

While applying the temperature cycle, the excitation light from the light source 450 is illuminated to the reaction cell 390. When the gene has a fluorescent dye intercalated within two chains, absorbed energy of the light from the light source 450 is transferred to the fluorescent dye (energy transfer). As a result, the fluorescent dye is excited and generates fluorescence. In other words, when the target gene is present in the sample, the intensity of the generated fluorescence increases as the target gene is amplified. Thus, by monitoring the intensity of fluorescence emitted from the reaction cell 390 with the photodetector 181, the presence or absence of the target gene can be detected in real time using the analysis chip 300 shown in FIG. 7. Further, with the analysis chip 300 set in a vertical posture in the analysis apparatus 400, even if a part of the reaction materials evaporates during the temperature cycle and the vapor resides in an upper portion of the reaction cell 390, the side surface of the reaction cell 390 emitting the fluorescence to be detected is not clouded by the vapor. This leads to an advantage that detection sensitivity is not reduced. (Step 112)

Finally, after the completion of the analysis, the analysis chip 300 is taken out of the analysis apparatus 400 and is then discarded. Because neither posttreatments of the sample and the reagents nor the operation of washing the reaction and detection components are required, the analysis can be performed in a simple and prompt manner. (Step 113)

According to the first embodiment, as described above, by using the collection chip 200 and the analysis chip 300 in combination with the collector 100 and the analysis apparatus 400, the steps from the germ collection to the germ detection can be automatically performed within the two kinds of small-sized chips 200 and 300. Since the treatment of the germ spores and the gene extraction step require no manual operations, the test operator can perform the analysis with safety regardless of skill. Further, since each of the chips 200 and 300 includes no mechanical parts such as valves, the chips 200 and 300 suitable for disposal after use can be provided. Also, as a result of forming the reaction cell and the channels by the photolithography to have smaller volumes, the amounts of regents used can be reduced and the cost can be cut correspondingly. Another advantage is in enabling faster temperature control, quicker mixing, and more uniform reaction to be realized. In addition, by containing the reagents only for one inspection in the disposable chips 200 and 300 in advance and providing the chips 200 and 300 to users in cold or frozen storage, it is possible to provide bio-analysis chips capable of detecting the target gene in a very simple and prompt manner and of being discarded after the analysis together with the reagents.

### Second Embodiment

In the first embodiment, the analysis chip 300 has one reaction cell 390 formed therein. To be adapted for the case of analyzing a plurality of targets, however, the reaction cell 390 may be formed in plural number. In such a case, because primers are required in one-to-one relation to the kinds of germs to be inspected, the reservoir for storing the gene amplification reagent A, which contains the primer, is also required in corresponding plural number. Further, it is required to change the illuminated position of the excitation light from the light source 450 depending on the location of each reaction cell 390 so that reactions in the plurality of reaction cells 390 can be detected by using the excitation light from one light source. However, the second embodiment is advantageous in that plural kinds of germs can be inspected in one analysis chip at the same time.

### Third Embodiment

The first embodiment has been described above as providing one chip mount unit on which the collection chip 200 and the analysis chip 300 are selectively set. In order to simultaneously perform the treatments for the collection chip 200 and the reaction cell 390 in parallel, however, two chip mount units may be provided in one apparatus so that the collector and the analysis apparatus are combined into the one apparatus. Because the optical detection system is not required in the treatment steps in the collection chip 200, the analysis apparatus 400 can be modified so as to include the two chip mounts unit by providing the fluid system and the temperature control system in dual. Although the size of that one apparatus is somewhat increased, the time required for processing a large number of samples can be cut by performing the treatments for the collection chip 200 and the reaction cell 390 at the same time.

### Fourth Embodiment

The fourth embodiment differs from the first embodiment in that a piezoelectric element, e.g., a quartz oscillator or a surface acoustic wave element, is attached to the bottom of the analysis chip 300. A piezoelectric element has a property of quantitatively converting the magnitude of a weight attached on its electrode to the change of oscillation frequency, and therefore it is widely used as means for continuously measuring a small mass change in a reaction atmosphere. Various kinds of nucleotides each having the known predetermined base sequence are fixed to the piezoelectric element. The fixing of the nucleotides is preferably performed as follows. First, a glass thin film is formed on the electrode of the piezoelectric element by sputtering or vapor deposition, for example. Glass used here preferably contains, as a main ingredient, SiO₂ that shows the best adhesion with an electrode material such as chromium or titanium. By adding aminopropyltrimethoxysilane (APS) to the glass thin film and baking it at temperature of about 120-160° C., amino groups are fixed on the surface of the glass thin film. Each of the electrode and the glass thin film has a thickness preferably in the range of 0.1-1 μm. The reason is that if the thickness of each of the electrode and the glass thin film exceeds 1 μm, the frequency response of the piezoelectric element deteriorates. Thereafter, the nucleotides are applied to the glass thin film coated with the amino groups, and the piezoelectric element is left to stand at temperature of 37° C. and humidity of 90% for 1 hour in a thermo-hygrostat. An ultraviolet ray of 60 mJ/cm² is then irradiated to the piezoelectric element by using a UV cross linker, whereby the nucleotides are firmly fixed to the piezoelectric element.

In the fourth embodiment, the steps from the sample collection to the gene extraction are the same as those in the first embodiment. When genes introduced to the reaction cell 390 are heated to about 94° C. by the temperature control mechanism 415, each gene is thermally denatured into a single chain. This single chain is bound to the nucleotide fixed at the chip bottom, whereupon the oscillation frequency of the piezoelectric element is changed. Thus, the gene sequence which is complementary to the sequence of the fixed nucleotides can be read by measuring the change of the oscillation frequency.

When the piezoelectric element is used in a liquid, the oscillation frequency is changed by 15-30 Hz if the liquid temperature is changed 1° C. Therefore, accurate control of the liquid temperature is essential in the fourth embodiment. However, the fourth embodiment is advantageous in that no gene amplification reagents are required and the detection time is shortened because of no necessity of the temperature cycle.

What is claimed is:

1. A bio-analysis chip comprising a collection member for collecting microbes in the atmosphere with the microbes adhering to said collection member, and a substrate in the form of a thin plate on which said collection member is mounted,
wherein said substrate has a collection member receiving section in which said collection member is received, and a plurality of reagent reservoirs in which reagents for treating and analyzing the microbes are stored,
said plurality of reagent reservoirs being connected to said collection member receiving section.

2. The bio-analysis chip according to claim 1, wherein said collection member receiving section is in the form of a shallow recess having an opening through which the atmosphere containing the microbes is introduced, said collection member being received at a bottom of said recess opposite to the opening, and the opening is enclosed by a sealing material in a state of the microbes adhering to said collection member.

3. The bio-analysis chip according to claim 2, wherein an upper part of said collection member receiving section is communicated with an air hole opened to the outside in a state of the reagents being fed from said reagent reservoirs to said collection member receiving section.

4. The bio-analysis chip according to claim 1, wherein said collection member receiving section is arranged in a central area of said substrate, and said plurality of reagent reservoirs are arranged in surrounding relation to said collection member receiving section.

5. The bio-analysis chip according to claim 4, wherein said plurality of reagent reservoirs are in the form of elongate fine channels each having one end connected to said collection member receiving section and the other end connected to a chip port which serves as a junction port communicating with the outside.

6. The bio-analysis chip according to claim 1, wherein said collection member is made of agar with a concentration of 2-5%, to which are added alcohols with a concentration of 40-80%.

7. The bio-analysis chip according to claim 1, wherein said bio-analysis chip is used to analyze genes of germs forming spores, and said plurality of reagent reservoirs include a reagent reservoir for storing a germination accelerator, a reagent reservoir for storing a cell lysin, and a reagent reservoir for storing chaotropic ions.

8. The bio-analysis chip according to claim 1, wherein said substrate and said collection member are each made of a material capable of being incinerated.

9. A bio-analysis system comprising a collector, bio-analysis chips constituted as a collection chip and an analysis chip, and an analysis apparatus,
wherein said collection chip comprises a collection member for collecting microbes in the atmosphere with the microbes adhering to said collection member, and a collection-chip substrate in the form of a thin plate on which said collection member is mounted;
said collection-chip substrate has a collection member receiving section in which said collection member is received, and a plurality of reagent reservoirs in which reagents for treating and analyzing the microbes are stored;
said collector causes the microbes in the atmosphere to collide against and adhere to said collection member when said collection chip is set in said collector;
said analysis chip includes an analysis-chip substrate in the form of a thin plate;
said analysis-chip substrate has a sample pool for storing a sample having been subjected to pretreatment on said collection chip, and a plurality of reagent reservoirs for storing reagents used to perform posttreatment for analysis of the microbes; and
said analysis apparatus causes the pretreatment to be performed in said collection chip when said collection chip is set in said analysis apparatus, and causes the postreatment to be performed in said analysis chip when said analysis chip is set in said analysis apparatus.

10. The bio-analysis system according to claim 9, wherein said collector and said analysis apparatus are combined into one unit.

11. The bio-analysis system according to claim 9, wherein said collector comprises a nozzle for sucking the atmosphere, a collection chamber provided in the outlet side of said nozzle, a mechanism for detachably attaching said collection chip, and an exhaust portion through which air sucked into said collection chamber is exhausted to the side opposite to said nozzle, said nozzle having a hole with an inner diameter of 4-15 mm.

12. The bio-analysis system according to claim 9, wherein said analysis chip is arranged in a vertical posture in said analysis apparatus.

13. The bio-analysis system according to claim 9, wherein the plurality of reagent reservoirs in said collection chip have respective one ends connected to said collection member receiving section and respective other ends connected to a plurality of chip ports which serve as junction ports communicating with the outside; and
said analysis apparatus includes an analysis-apparatus base plate having a plurality of channels connectable to the chip ports of said collection chip, and a fluid supply mechanism for selectively supplying a fluid from the plurality of channels in said analysis-apparatus base plate to the chip ports of said collection chip through valves.

14. The bio-analysis system according to claim 13, wherein the plurality of reagent reservoirs in said analysis chip are connected to the plurality of chip ports which serve as the junction ports communicating with the outside, said analysis-apparatus base plate has a plurality of channels connectable to the chip ports of said analysis chip, and said fluid supply mechanism selectively supplies the fluid from the plurality of channels in said analysis-apparatus base plate to the chip ports of said analysis chip through said valves.

15. A bio-analysis method comprising the steps of:
setting, in a collector, a bio-analysis chip including a collection member receiving section in which a collection member is received, and a plurality of reagent reservoirs in which reagents for treating and analyzing microbes are stored, and operating a blowing mechanism in said collector such that the microbes in the atmosphere are caused to collide against and adhere to said collection member; and
setting said bio-analysis chip containing the adhered microbes in an analysis apparatus, and operating a fluid feeding mechanism in said analysis apparatus such that the reagents in the plurality of reagent reservoirs in said bio-analysis chip are successively supplied to said collection member, thereby treating and analyzing the microbes.

16. A bio-analysis method comprising the steps of:
setting, in a collector, a first bio-analysis chip including a collection member receiving section in which a collection member is received, and a plurality of reagent reservoirs in which reagents for performing pretreatment for analysis of microbes are stored, and operating a blowing mechanism in said collector such that the microbes in the atmosphere are caused to collide against and adhere to said collection member;
setting said first bio-analysis chip containing the adhered microbes in an analysis apparatus, and operating a liquid feeding mechanism in said analysis apparatus such that the reagents in the plurality of reagent reservoirs in said first bio-analysis chip are successively supplied to said collection member, thereby performing the pretreatment for the analysis of the microbes; and
setting, in said analysis apparatus, a second bio-analysis chip including a sample pool, a plurality of reagent reservoirs in which reagents for performing posttreatment for analysis of the microbes are stored, and a reaction cell, in a state of said sample pool being filled with the sample having been subjected to the pretreatment for the analysis, and operating the liquid feeding mechanism in said analysis apparatus such that the sample in said sample pool and the reagents in said plurality of reagent reservoirs are successively supplied to said reaction cell, thereby performing the posttreatment for the analysis of the microbes.

17. The bio-analysis method according to claim 16, wherein the bio-analysis method is used to analyze genes of germs forming spores,
the pretreatment performed in said first bio-analysis chip comprises the steps of applying a germination accelerator and bringing cell walls into lysis, and
the posttreatment performed in said second bio-analysis chip comprises the steps of binding the genes after the lysis of the cell walls to a gene binding carrier, washing a bound substance of the genes and the gene binding carrier, eluting the genes from the gene binding carrier, and detecting the eluted genes.
